(19) United States
(12) Patent Application Publication
(10) Pub. No.: US 2003/0120217 A1
(43) Pub. Date: Jun. 26, 2003

(54) Methods and devices for sclerotherapy

(76) Inventor: R. Patrick Abergel, Santa Monica, CA (US)

Correspondence Address: PERKINS COIE LLP, POST OFFICE BOX 1208, SEATTLE, WA, 98111-1208, US

(21) Appl. No.: 10/074,348

(22) Filed: Feb. 11, 2002

Related U.S. Application Data
(63) Continuation-in-part of application No. 10/029,321, filed on Dec. 21, 2001.

(51) Int. Cl. A61M 1/00

(52) U.S. Cl. 604/191

(57) ABSTRACT

In a method for sclerotherapy for treating varicose veins, a flushing solution, such as sterile saline solution, is initially injected into the vein or vessel being treated. The flushing solution displaces or flushes out blood from the treatment site of the vessel. A sclerosing agent is then injected into the treatment site. The displacement of blood before introduction of the sclerosing agent reduces complications. A syringe assembly useful for performing the method has first and second reservoirs sealed off from each by an end cap. The end cap is removed just before use. A needle is attached and is connected to both reservoirs. Flushing solution is delivered from the first reservoir followed by sclerosing solution delivered from the second reservoir, without removing the needle from the vessel. A system or kit for combining and holding two syringes, to provide two different injectants into the same injection site, includes a collar holding two (or more) syringes together. A manifold provides a common outlet into a single needle.

This Application is a Continuation-In-Part of U.S. patent application Ser. No. 10/029,321, filed Dec. 21, 2001 and now pending.

### FIELD OF THE INVENTION

The field of the invention is sclerotherapy. More specifically, the invention relates to the treatment of spider and varicose veins via sclerotherapy. The invention further relates to a novel syringe assembly useful in sclerotherapy, as well as in other medical applications.

### BACKGROUND OF THE INVENTION

Spider veins or telangiectasias of the legs are common conditions, especially among women. With this condition, small dark-colored veins form on the legs, just underneath the skin surface. These types of veins can form anywhere on the legs between the thigh and ankle. They often have a web or sunburst pattern, but may also be formed as short, somewhat random line segments. In many cases, spider veins are largely unnoticeable, when localized in a small area. However, when larger areas of skin are affected, spider veins can be perceived as having a detrimental appearance on the skin.

Varicose veins are larger veins, in comparison to spider veins. Varicose veins may protrude or be raised above the skin surface. They typically have a blue or purple color. A varicose vein generally contains stagnant or refluxing blood, which is out of circulation. Consequently, a varicose vein no longer functions to channel blood flow back to the circulatory system or the heart. Larger veins have valves which maintain blood flow in the forward direction. If the valves fail, blood accumulates under pressure, causing the veins of the leg to engorge. These varicose veins often appear as bulging, and have a rope-like or thread-like appearance. In more severe cases, these vascular disorders can result in aching, throbbing, swelling, or other conditions requiring medical treatment. Moreover, many patients having varicose veins, even without these symptoms, become distressed by the appearance of the varicose veins. Consequently, various treatments have been developed for both medical and cosmetic reasons. These treatments include surgery for severe cases, as well as sclerotherapy, typically used for smaller varicose veins closer to the skin surface. In the past, sclerotherapy has been performed by injecting a sclerosing agent into the vein. This non-surgical procedure destroys the varicose vein by irritating the vein wall, and causing the vein to close up. Procedures using ultrasound, or an electrosurgical electrode in combination with sclerotherapy have also been proposed. In general, sclerotherapy is a proven, safe, and effective technique.

Notwithstanding the successes of sclerotherapy, complications can occur with these treatments. One such complication is ulceration. This complication results when a sclerosing solution is inadvertently injected intra-dermally or into surrounding tissue, rather than into the intended injection site in a vein. The sclerosing solution delivered outside of the vein can cause ulceration of the skin and surrounding tissue. As it may be difficult to consistently position the needle into the vein, this type of ulceration is a common complication. Hyperpigmentation is another complication. This complication results due to leakage of a blood component, hemosiderin pigment, from the damaged blood vessel or vein. Another complication is mat-like telangiectasia, which is the appearance of new, small blood vessels. Mat-like telangiectasia is believed to result from injection of an excessive amount of sclerosing solution.

Accordingly, it is an object of the invention to provide improved methods and devices for treating spider and varicose veins via sclerotherapy.

In certain medical procedures, it is advantageous to be able to inject different solutions into a single injection site, either simultaneously, or sequentially. Currently, this generally may require two separate injections. As a result, the two injections may not be located at the same location. In addition, two separate injections requires more time to provide, consumes more syringes and needles (generating more medical waste), and causes more pain and risk of infection to the patient.

Accordingly, it is also an object of the invention to provide a novel syringe assembly useful for injecting two separate solutions in a single procedure.

### SUMMARY OF THE INVENTION

To these ends, in a first aspect, a method for treating a spider vein or a varicose vein via sclerotherapy includes injecting a flushing solution into the vein, to flush out blood from the section of the vein treated. A sclerosing solution is then injected into the blood vessel. The flushing solution and sclerosing solution are preferably injected from a single hypodermic needle. This allows both the flushing and sclerosing solutions to be injected sequentially at the same location and via a single injection or piercing of the skin and vein.

In a second aspect of the invention, a syringe assembly for providing sclerotherapy has two separate reservoirs, chambers, or syringes. The first reservoir contains a flushing solution, preferably sterile saline solution. The second reservoir contains a sclerosing agent. A needle is attached at one end of the syringe assembly. Both reservoirs are connectable into the preferably 30-gauge needle. Each reservoir has a separate plunger.

In use, in a third aspect of the invention, the needle is positioned in the vein or vessel to be treated. The plunger in the first reservoir is pressed, injecting the flushing solution into the vessel. Blood is flushed or displaced from the injection site. The second plunger is then pressed to inject the sclerosing agent into the vessel. By flushing the blood from the vessel, prior to injecting the sclerosing solution, the potential for blood leakage is greatly reduced. In addition, flushing the vessel with saline reduces the need to inject excessive amounts of sclerosing solution, thereby minimizing the potential of the mat-like telangiectasia complication.

In a fourth aspect of the invention, the potential for inadvertently causing skin ulcerations by injecting a sclerosing solution at an improper location is reduced or eliminated. A sterile saline or other flushing solution is injected first into the injection site. The physician visually observes the injection site. If the vein disappears or tends to fade from view, the needle is properly positioned in the vein, and has flushed out the blood. The sclerosing solution is then injected into the same site via the same needle, without withdrawing the needle from the site. On the other hand, if the vein appearance remains largely unchanged after the flushing solution is injected, the physician then has a visual indication that the needle is not properly positioned in the vein. The needle is then withdrawn and relocated, and the visual observation procedure is repeated. This method avoids inadvertent injection of sclerosing solution into tissue outside of the vein, and the potential complications, such as skin ulcerations, which may accompany such events.

In a fifth and separate aspect, a novel syringe assembly has two separate reservoirs connecting to a single needle. The liquid contents of the two reservoirs are separated from each during storage. Consequently, the syringe can be advantageously pre-filled with two different injectant solutions, and then optionally sealed in a package until use. The liquid contents are contained or sealed within the reservoirs by a plunger seal on a plunger towards the back end of the syringe assembly, and by an end cap or closed off needle at the front end. In use, an end cap is removed from the syringe assembly and a needle is attached, with the bore of the needle connecting into both reservoirs. Alternatively, a needle may be attached to the syringe assembly during or after manufacture, and no end cap is used. A needle tip protector/seal may optionally be pushed on to the tip of the needle, to avoid piercing packaging and needle stick incidents, and to prevent leakage from the reservoirs. The syringe assembly provides for improved sclerotherapy procedures, and may also be used for other procedures as well.

In a sixth and separate aspect of the invention, a syringe holding system includes a collar and a manifold, which allow standard syringes to be combined into a unit which is easily handled, for delivering different injections into the same injection site.

Other and further objects and advantages will appear. The invention resides as well in subcombinations of the methods and devices shown and described.

### BRIEF DESCRIPTION OF THE DRAWINGS

FIG. 1 is a perspective view of a syringe assembly for use in sclerotherapy, with a needle attached to the syringe assembly.

FIG. 2 is a section view taken along line 2-2 of FIG. 1.

FIG. 3 is enlarged perspective view of the end section of an alternative syringe design.

FIG. 4 is a perspective view of the syringe assembly of FIG. 1 with an end plug attached to the syringe assembly.

FIG. 5 is an enlarged perspective view of the end tube and end cap shown in FIG. 4.

FIG. 6 is an enlarged perspective view of the cap shown in FIGS. 4 and 5.

FIG. 7 is a side view, in part section, of an end cap on the end tube.

FIG. 8 is a schematic view of the needle shown in FIGS. 1 or 2 inserted into a vessel or vein.

FIG. 9 is an enlarged partial section view of an alternative syringe assembly similar to the syringe assembly shown in FIG. 7, and having an end tube divider plate extending from the reservoir outlets to the front end of the syringe assembly.

FIG. 10 is a perspective view of an alternative end cap for use with the syringe assembly shown in FIG. 9.

FIG. 11 is an exploded perspective view of alternative two syringe fixture system.

FIG. 12 is a perspective view of the assembled system shown in FIG. 11, with the arms on the collar assembly in an open position for installation or removal of syringes.

FIG. 13 is a perspective view of the system shown in FIGS. 11 and 12, with the arms in a closed position.

FIG. 14 is a side view of the manifold shown in FIGS. 11-13.

FIGS. 15-17 are alternative collar assembly designs.

FIG. 18 is a perspective view of another alternative to syringe holding or fixturing system.

FIG. 19 is a perspective view showing assembly of the syringe system of FIG. 18.

FIG. 20 is an exploded perspective view of the manifold shown in FIG. 18.

FIG. 21 is a perspective view of the valve body shown FIG. 20.

FIG. 22 is a side view of the syringe system of FIG. 18, with the valve body in a first position, for delivering an injectant from a first syringe while preventing back-flow into the second syringe.

FIG. 23 is a side view of the syringe system of FIG. 18, with the valve body in a second position, to deliver an injectant from a second syringe, while preventing back-flow into the first syringe.

FIG. 24 is an enlarged side view of an alternative modification of the design shown in FIG. 1.

FIG. 25 is an exploded perspective view of the syringe unit including the modification shown in FIG. 24.

### DETAILED DESCRIPTION

The invention provides sclerotherapy methods having reduced risk of complications. The methods involve flushing or displacing blood from the vein or vessel, before injecting a sclerosing solution into the vessel. Injection of a clear flushing solution, such as sterile saline displaces blood from the vein. The vein can then be difficult or impossible for the physician to see. Consequently, injecting the sclerosing solution through the same needle, at the same injection site, avoids the need to find the vein, after the flushing solution is injected. Consequently, the methods are more advantageously performed using a syringe assembly which can deliver both solutions with a single injection, through a single needle. This reduces the number of injections required. In addition, it ensures that the flushing and sclerosing solutions are injected at the same location, while avoiding the difficulties of finding the vein after injection of the flushing solution.

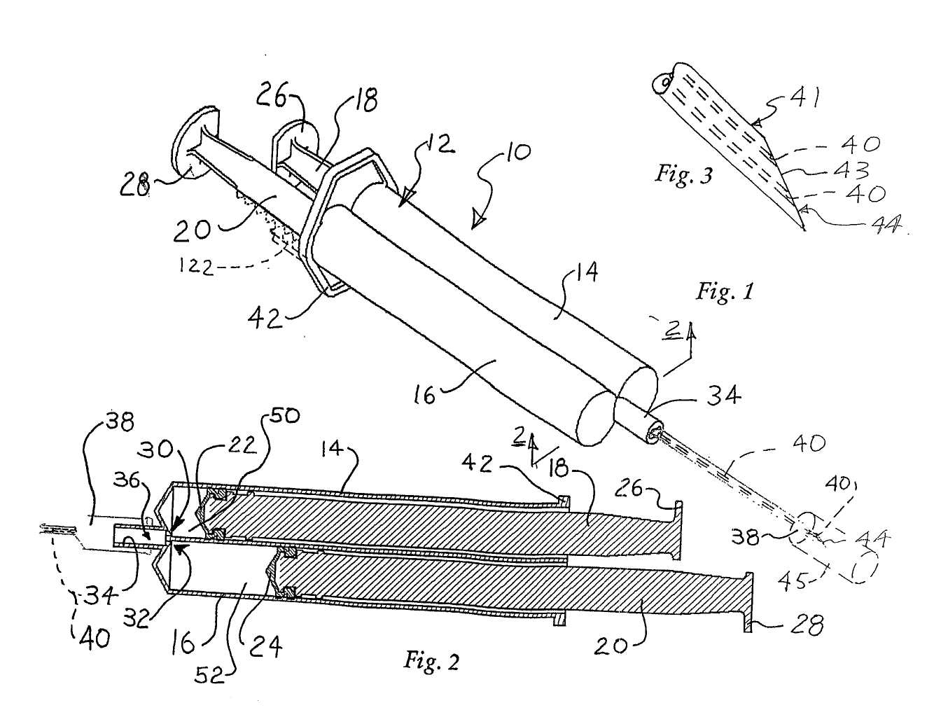

FIGS. 1 and 2 show a preferred syringe assembly for performing the methods described. As shown in FIG. 1, the syringe assembly 10 has a body 12, preferably formed as a single molded plastic unit. The body 12 includes first and second barrels, chambers or reservoirs 14 and 16. A first plunger 18 having a first end seal 22 is slidably positioned within the first reservoir 14. Similarly, a second plunger 20 having a second end seal 24 is slidably positioned within the second reservoir 16.

The first plunger 18 has a first cap 26 and the second plunger 20 has a second cap 28. The caps 26 and 28 are D-shaped, so that they can pass by each other. In use, the positions of the end caps 26 and 28 also provide a convenient visual and tactile indication of the relative volumes of fluids injected. For example, when they are aligned, the user knows that equal volumes have been injected. A hollow end tube 34 extends from the bottom or front end of the body 12. A bore or opening 36 in the end tube 34 connects into the first reservoir 14 through a first outlet 30. Similarly, the bore 36 in the end tube 34 also connects to the second reservoir 16 through a second outlet 32. The outlets 30 and 32 connect the reservoirs 14 and 16 directly into the bore 36. No valves or other flow control features are needed or used in this embodiment.

A hypodermic needle 38 having a needle bore 40 is attachable to the end tube 34 using e.g., a Luer fitting. Of course, other types of needles and fittings, bayonet, screw threads, etc., may also be used. A finger flange 42 is advantageously provided at the back or top end of the body 12. The first reservoir 14 is preferably filled with a flushing solution 50, preferably sterile saline solution. The second reservoir 16 is preferably filled with a sclerosing solution 52. The sclerosing agent may be sodium morruhate, sodium tetradecylsulfate, polilocanol, chromated glycerine, polyiodine iodine, hypotonic saline, Lauromacchogal, Abtysisclerol or other known sclerosing agent, in solution.

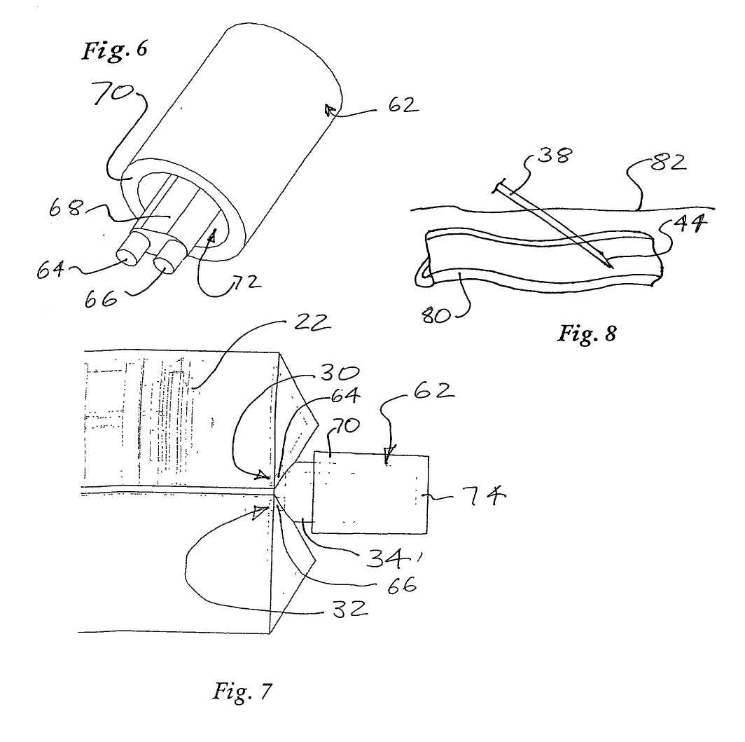

The syringe assembly 10 may advantageously be pre-filled with the solutions 50 and 52, with an end cap 62 on the end tube 34, to prevent leakage during shipment and storage, and to maintain sterility. An over package, envelope, or container 90, may optionally be provided, enclosing the syringe assembly 10, to further maintain sterility of the reservoir contents. With the reservoirs 14 and 16 filled, the plungers 18 and 20 are fully withdrawn. FIGS. 1 and 2 show the plungers at intermediate positions, for purposes of illustration. As shown in FIGS. 5-7, the end cap 62 has plugs 64 and 66 on a neck 68. The cylindrical body 70 of the end cap 62 surrounds, and is spaced apart from the neck 68 via a gap 72. The neck is attached to the front end or surface 74 of the end cap 62. The end cap 62 makes a friction fit onto the end tube 34, with the end tube 32 sliding into the gap 72. The plugs 64 and 66 move into and plug the outlets 30 and 32. Consequently, with the end cap 62 in place, the contents of the reservoirs 14 and 16 are sealed from the environment, and from each other. Providing the syringe assembly 10 as a prefilled unit avoids the need for the physician to separately fill the reservoirs.

Referring to FIG. 2, although there is a direct connection between the first and second reservoirs 14 and 16 and the bore 36 in the end tube 34 via the first and second outlets 30 and 32, the outlets 30 and 32 are plugged by the plugs 64 and 66 during storage. Any mixing between the solutions 50 and 52 after the end cap 62 is removed and the needle 38 attached, is inconsequential due to the relatively small size of the outlets 30 and 32, the flow characteristics through the syringe assembly, and the short duration of use of the syringe assembly after the end cap is removed. As no valves or other flow control devices are needed in this embodiment, injection of the solutions 50 and 52 is quick and simple. The syringe assembly itself is also a simple and inexpensive design.

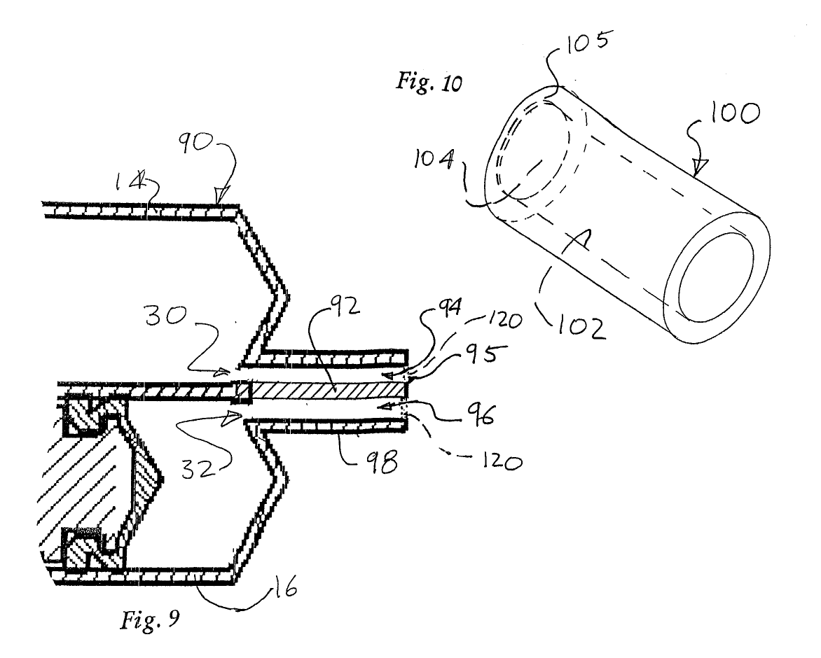

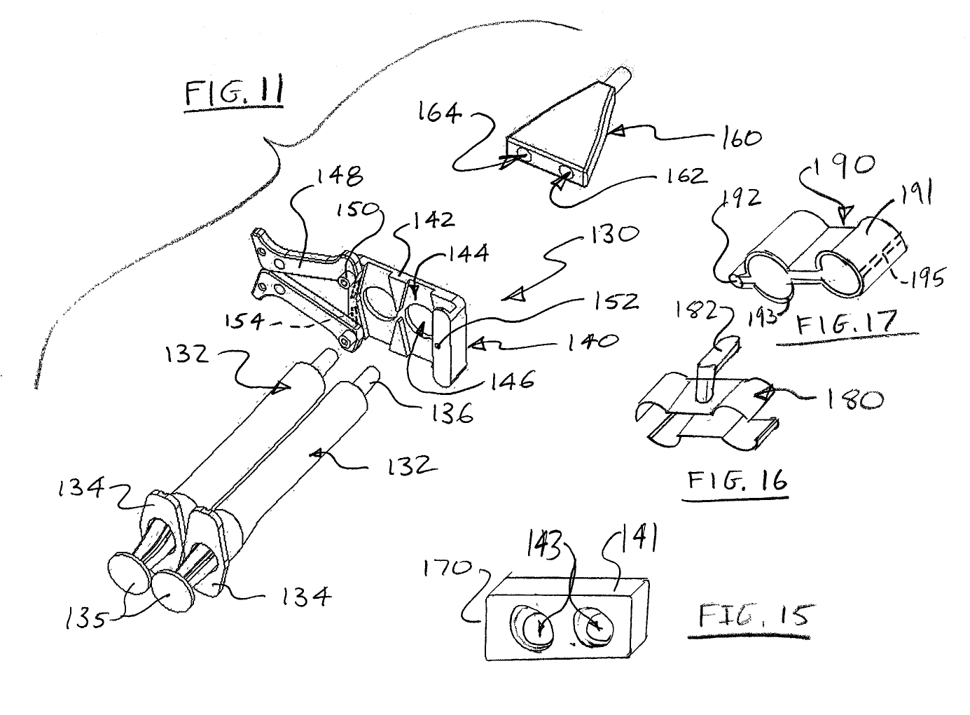

As shown in FIGS. 9 and 10, in an alternative syringe assembly 90, an end tube divider 92 extends from the outlets 30 and 32 to the very front end surface 95 of the end tube 98. This divides the end tube 98 into two separate bores 94 and 96. The end tube 98 preferably has a slightly tapering or conical outside wall. The end cap 100 shown in FIG. 10 has a complimentary inner wall 102. When the end cap 100 is pushed on to the end tube 98, it remains in place via the mating of the complimentary tapered surfaces and friction. The bottom end 105 of the end cap 100 contacts the front end surface 95 of the end tube 98 and seals off both of the separate bores 94 and 96. This prevents leaking or mixing of the contents of the reservoirs. A resilient end cap pad or disk 104 may optionally be attached to the bottom surface of the end cap, to help seal the ends of the bores 94 and 96 when the end cap 100 is installed. The plunger seals 22 and 24 seal off the back end of the reservoirs.

The syringe assembly may also be used to store and/or inject other combinations of solutions. In one such embodiment, the first reservoir contains a heparin solution and the second contains a saline solution.

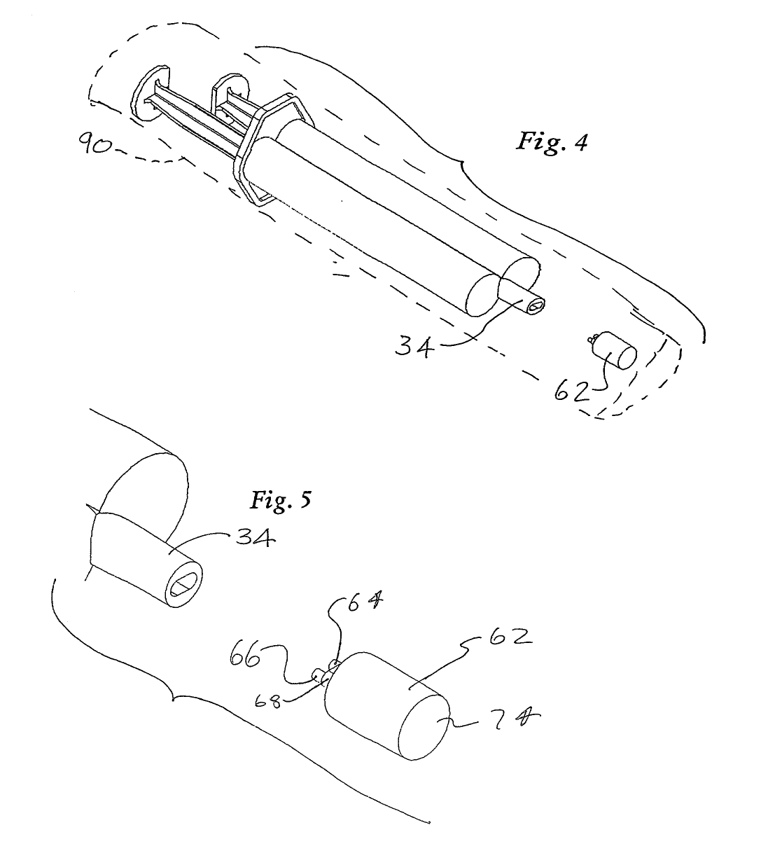

The needle 38 is preferably a 30-gauge needle. The needle preferably has a single lumen or bore 40 which connects to the outlets 30 and 32. This allows the single needle to deliver both solutions, while having a small diameter, and piercing only a small opening in the skin and vein. The needle 38 may be made part of, or be provided already attached to, the syringe assembly, with the syringe assembly/needle combination optionally provided with the package 90. As shown in dotted lines in FIG. 1, a needle cap 45 may be pushed or attached on to the tip of the needle. The needle cap helps to prevent piercing of the packaging, needle stick incidents, and leakage of the contents of the reservoirs out through the needle bore. The needle cap 45 may be resilient or rubber material. As the outlets 30 and 32 are both open into the bore of the needle, with this design, the contents of the reservoirs, over sufficient time, may diffuse into each other. If such diffusion must be entirely prevented, then the end cap 62 is installed in place of the needle, and the needle is installed only just before use. Alternatively, a needle 41 having two bores 40 may be used, as shown in FIG. 3, and with each bore separately connecting only to one of the reservoirs. With this design, separate flow paths from the outlets and through the tube 34 and needle 41 to the needle tip 43 are provided. Hence, in most ordinary uses any mixing of the solutions before injection is prevented, even with the needle attached to the syringe assembly during manufacture. However, the dual bore needle 41 necessarily requires a diameter larger than the single bore needle 38. The needle cap 45 can be used on needle 41 as well. The syringe assembly 10 shown in FIGS. 1-2 and 4-7 is used by removing the end cap 62 and attaching the needle 38 to the end tube 34. The design concepts described above may also be applied to a syringe assembly having three or more reservoirs.

Referring to FIGS. 2 and 9, as there is an open pathway between the outlets 30 and 32, either in the end tube 34, or within the bore of the needle, in rare situations, inadvertent mixing of the contents of the reservoirs may occur. Specifically, if flow through the needle bore is restricted, and the sliding friction of the plunger end seals 22 and 24 is sufficiently low, and if one of the plungers 18 or 20 is rapidly pushed in, then some liquid may flow from one reservoir into the other reservoir, causing inadvertent mixing. When this occurs, pushing on one plunger, e.g., 18, causes the other plunger 20 to move back out of the reservoir 16.

This result can be avoided in several ways. One way is to have the needle bore diameter (the I.D. of the needle) be larger than I.D. of the outlets 30 and 32. This will generally result in a flow resistance or pressure drop of flow through the needle bore, that is substantially less than flow resistance through either outlet 30 or 32. Hence liquid flowing out of outlet 30 will not flow into outlet 32. Typical hypodermic needles used with the syringe assemblies 10 or 90 range from Gauge 35 to Gauge 16. Gauge 35 has an O.D. of 0.012 and an I.D. of 0.006 inches. Gauge 16 has an O.D. of 0.065 and an I.D. of 0.053 inches. The bore diameters in this range are then nominally 0.006-0.013 inches. Accordingly, if the outlets are 0.005 inches or less, back flow mixing can be avoided in most or all cases. Outlet diameters of 0.001-0.005 inches are preferred for this range of needles. While both outlets typically will have the same diameter, one may be larger, depending on the liquid content characteristics, purity requirements, etc.

Another alternative to prevent backflow mixing is use of one-way check valves or other flow restrictors 120, as shown in FIG. 9. The valves 120 prevent inflow into the reservoirs through the outlets 30 or 32. However, use of valves 120 requires that the reservoirs be filled from the top or back end, by removing the plungers, filling, and then replacing the plungers. A flow restrictor further increases flow resistance as a function of flow velocity, effectively limiting the speed of plunger movement and preventing back flow mixing.

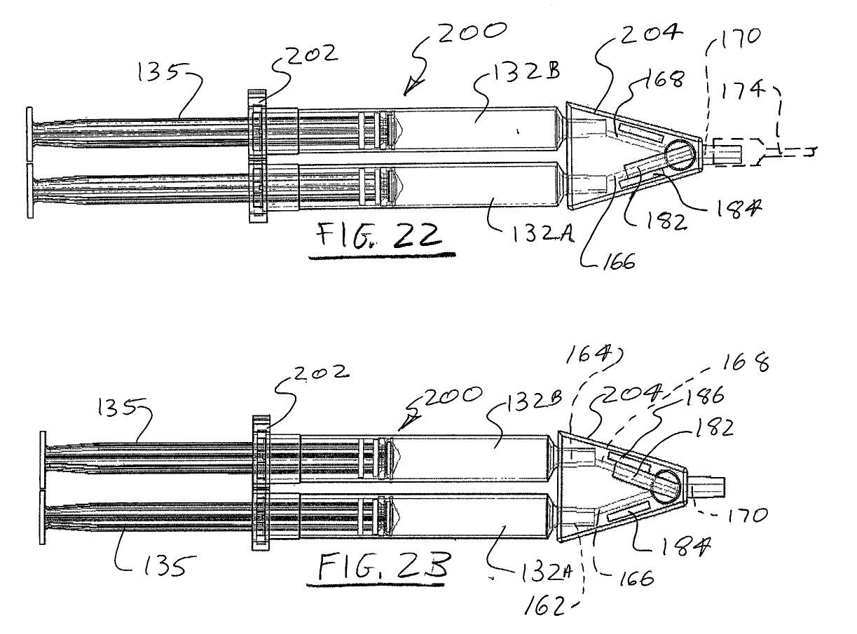

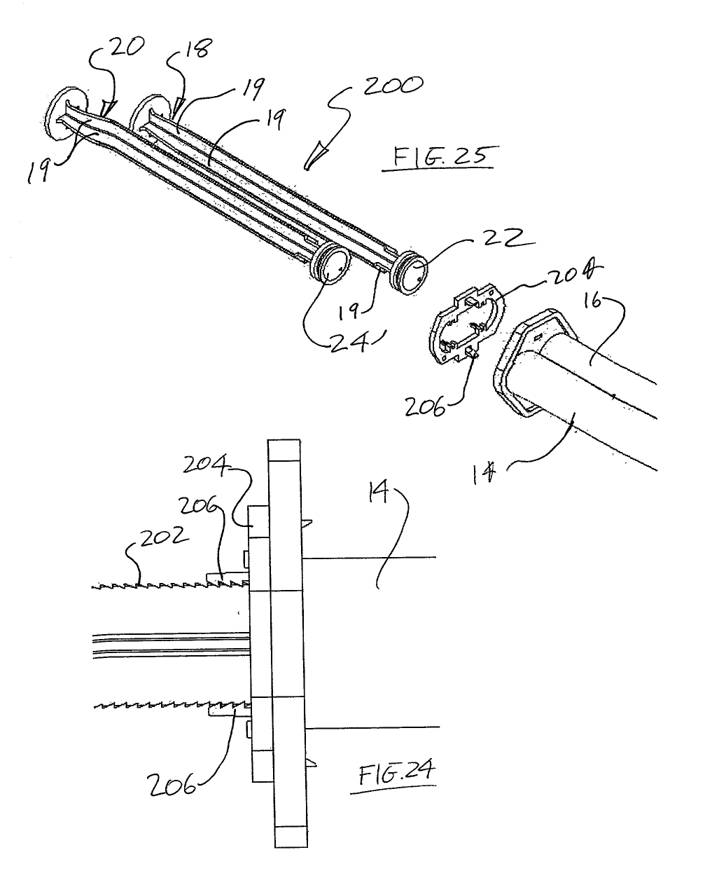

Another alternative to prevent backflow mixing is a one-way ratchet or brake 122 as shown in FIG. 1. The ratchet 122, when engaged, prevents backward movement of the plunger. The ratchet may be disengaged temporarily, to fill the reservoir by pulling the plunger back up through the reservoir. This design is shown in further detail in FIGS. 24 and 25. Referring to FIGS. 1, 24 and 25 in the alternative design 200, the plungers 18 and 20 have teeth 202, on one, two, three, or four of the ribs 19 of the plungers. A ratchet plate 204 is attached to the flange of the syringe. An arm 206 on the plate 204 engages the teeth 202. The arm 202 permits forward or inward movement of the plunger, but prevents outward or backward movement.

The plungers 18 and 20, modified to include the teeth 202, can only move in one direction when the arms 206 are engaged and hence prevent liquid from flowing from one syringe reservoir to the other. The arms 206 and/or teeth 202 may only cover a portion of the plunger, so that they may be disengaged by twisting the plungers near the end of travel. This allows the plungers to be withdrawn. The unit 200 can then be reused.

The plungers may be assembled at the factory with the teeth and arms disengaged. The end user can then fill the reservoirs by withdrawing the plungers, and then twist the plungers so as to engage the ratcheting mechanism formed by the engagement of the teeth and the arms. In a single use unit, the ratcheting teeth preferably extend over the whole length of the plunger, and the arms extend continuously or near continuously around the outside of the plungers, so that once engaged, it cannot be disengaged. Once the plungers reach the end of travel, the device is disposed of. The syringes may also be pre-filled with the ratchets engaged as well.

Referring back to FIGS. 1 and 2, in use for sclerotherapy, needle 38 is oriented with the angled tip surface 44 up and is inserted into the vessel or vein 80 to be treated. Once inserted, the angle or bevel surface 44 of the needle is facing up towards the skin 82, as shown in FIG. 8. The first plunger 18 is pressed in, injecting the flushing solution into the vessel. The flushing solution displaces blood in the vessel, moving the blood away from the treatment site. Typically, the vein will no longer be visible once the blood is displaced. If the physician observes little or no change in appearance in the vein, then the needle is not properly located in the vein. The needle is then withdrawn and inserted at another location in the vein, before the sclerosing solution is injected. Injection of sclerosing solution outside of the vein can cause skin ulcerations. On the other hand, inadvertent injection of a flushing solution such as a saline outside of the vein creates no such risk of complications The use of flushing solution first, along with the change in appearance (or disappearance) of the vein, reduces potential complications resulting from injecting sclerosing solution outside of the vein.

After the flushing solution is injected, and the physician observes the change in appearance of the vein, and with the needle 38 remaining in place in the vessel, the second plunger 20 is then pressed in, injecting the sclerosing solution 52 into the vessel. As the sclerosing solution 52 acts on the vessel walls, without significant presence of blood at the treatment site, complications, such as hyperpigmentation, are reduced.

The volume of flushing solution 50 and sclerosing solution 52 used with each procedure may vary depending on the size of the vessel and other factors.

After the treating physician determines that a sufficient amount of solutions 50 and 52 have been injected, the needle 38 is withdrawn from the vessel. A pressure dressing may be applied to the skin around the injection site. The needle may be relocated to another treatment site, on the same vessel, or on a different vessel. The reservoirs hold 1-8, 2-6 or 3-5 ml each. These volumes allow for multiple injections with a single syringe assembly 10. Ultrasound treatment may be used on the areas treated over the 24-78 hour period following the above-described methods of sclerotherapy. This helps to further reduce or avoid bruising or potential hyperpigmentation.

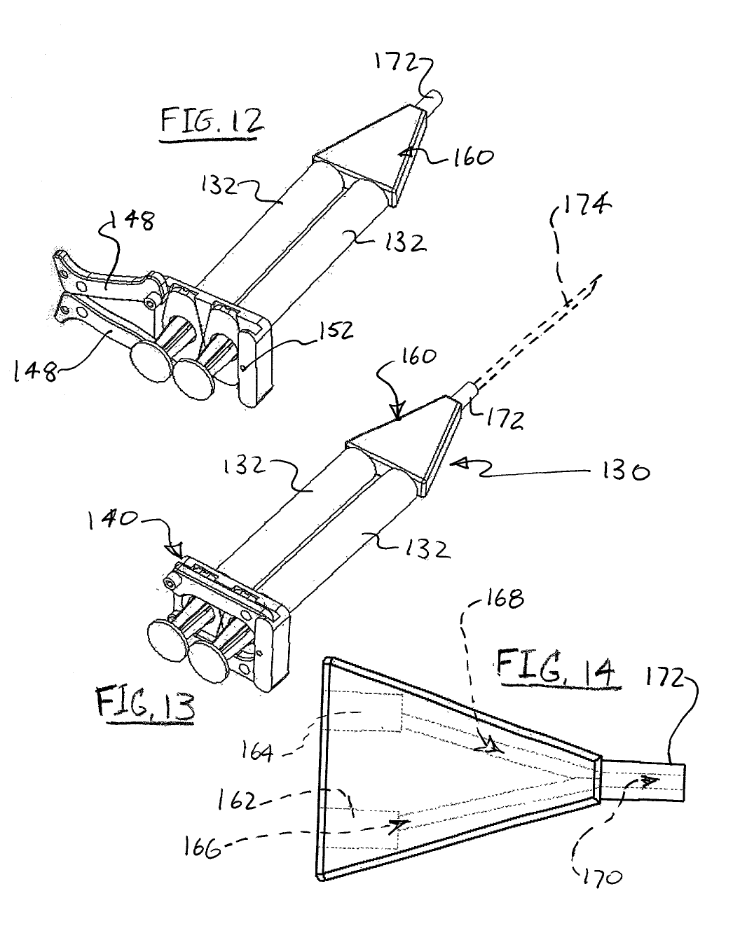

Referring now to FIGS. 11-14, a syringe fixture system 130 combines two standard syringes 132 using a collar assembly 140 and a manifold 160. The system 130 includes first and second syringes 132, the collar assembly 140, the manifold 160 and a needle 174 attachable to the manifold 160.

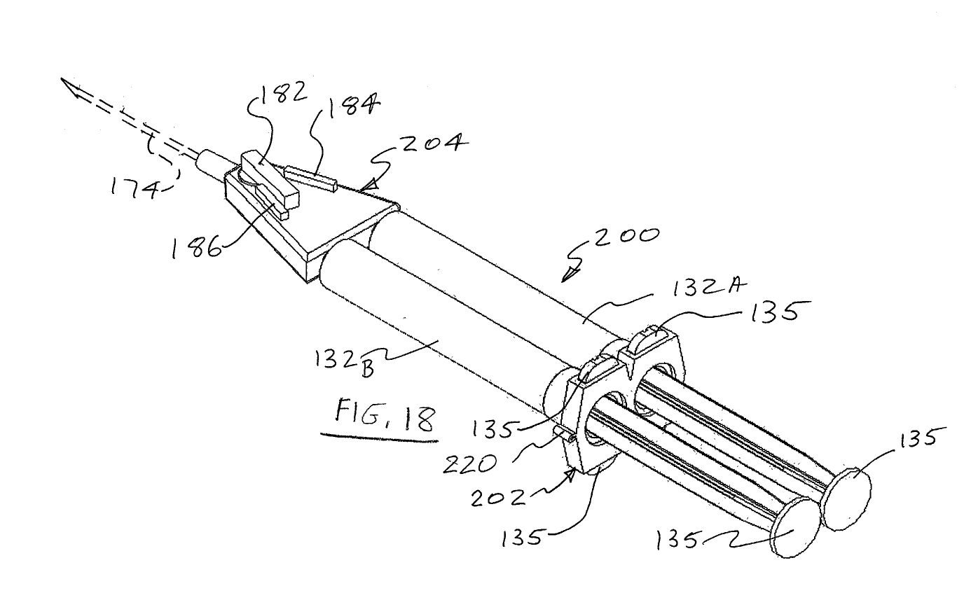

Referring to FIG. 11, the syringes 132 have a flange 134 and an end tube 136. The collar assembly 140 includes a collar plate 142 having first and second flange recesses 144, adapted to receive the flanges 134 of the syringes 132. Clearance holes 146 are aligned within each of the flange recesses. First and second arms 148 are pivotably attached at one side of the plate 142, for example, with cap screws 150. The arms 148 are pivotable or movable from the open position, shown in FIGS. 11 and 12 to the closed position, shown in FIG. 13. A detent 152 may be provided on the plate 142, to hold the arms 148 into the closed position shown in FIG. 13. Alternatively, or in addition, a spring 154 on the plate 142 may bias each of the arms 148 into the closed position. Preferably, the inside end surfaces of the arms 148 are curved to generally match the radius of the cylindrical barrels of the syringes 132.

The manifold 160 has first and second end tube openings 162 and 164, adapted to receive and engage with the end tube 136 on the syringes 132. Referring to FIG. 14, the first and second end tube openings 162 and 164 connected respectively into first and second bores 166 and 168, which join together into an outlet 170, at the front end of the manifold 160. The outlet 170 passes through a needle fitting 172 on the manifold 160.

In use, the arms 148 are moved and/or held into the opening position 148. First and second syringes 132 are placed into the collar assembly 140, with the flanges 134 of the syringes 132 positioned within the recesses 144, as shown in FIG. 12. Various standard syringes may be used. The arms 148 are then moved from the open position, shown in FIG. 12, to the closed position, shown in FIG. 13. The arms 148 are secured into the closed position by the detent 152 and/or the spring 154.

The manifold 160 is then pushed onto the syringes 132. Specifically, the end tube openings 162 and 164 are aligned with and engaged over the end tubes 136 of the syringes 132. The end tube openings 162 and 164 may have tapering or conical walls, to securely engage onto the end tubes 136, with a generally fluid-type fit. A needle 174 is then attached to the needle fitting 172 on the manifold 160.

In use, the syringes 132 are secured together into the single two syringe fixture unit 130, as shown in FIG. 13. Liquid from either syringe 132 may be injected via the single needle 174. Consequently, the two syringe fixture unit shown in FIG. 13 is advantageous for performing sclerotherapy, as described above.

The syringes 132 may be pre-filled with a liquid injectant, before they are secured together into the unit 130 shown in FIG. 13. Alternatively, the unit 130 shown in FIG. 13 may be formed with empty syringes. The syringes 132 are then filled with injectants by placing the needle 174 into the source of the liquid injectant, and then pulling back on the appropriate plunger 135.

The size and shape of the collar assembly 140 and flange recesses 144 may be changed as needed to accommodate syringes 132 having various sizes and shapes. The collar assembly 140 may be made of metal or plastic, and may be used as a disposable, or a reusable component. The liquid injectant moving out of the syringes 132 into the needle 174 pass through the passageways 166, 168 and 170, in the manifold 160. Accordingly, the manifold 160 is preferably made of a material which does not interact with the liquid injectant. The needle fitting 172 on the manifold 160 preferably uses standard syringe and needle fittings.

The system 130 may be provided as a kit, including all of the components shown in FIG. 13. Alternatively, since the syringes 132 and needle 174 are common and standard medical products, the system may also be provided without them, and include only the collar assembly 140 and the manifold 160.

The embodiment shown in FIGS. 11-14 is preferred for use in sclerotherapy, as described above, as it holds a first syringe containing sterile water, and a second syringe holding a sclerosing injectant. Of course, the collar assembly 140 and manifold 160 may also be designed to hold 3, 4, or more syringes, although 2 is preferred.

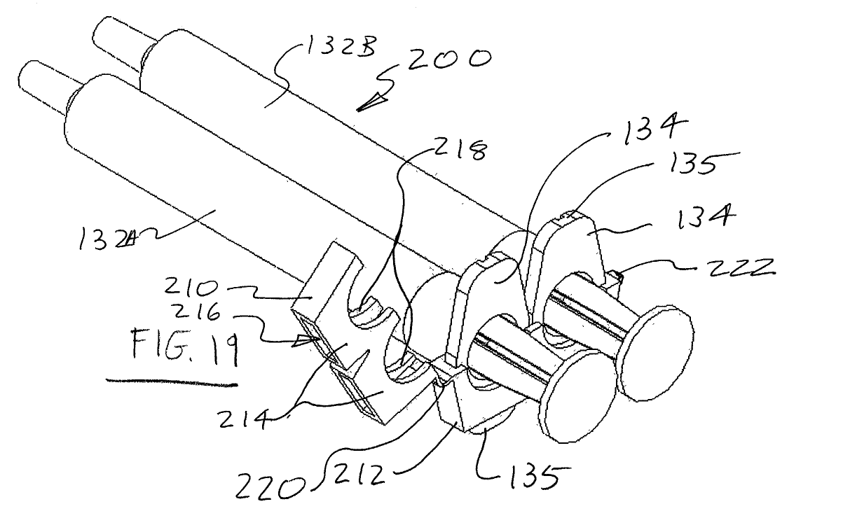

FIGS. 15-17 show alternative collar assemblies. FIG. 15 shows a perspective view of a simplified collar assembly 141 which may be used in place of the collar assembly shown in FIGS. 11-13. The collar assembly 141 is simply a plate or block having clearance holes 143 for the cylindrical bodies of the syringes 132. The holes 143 may be straight or with a slight conical taper. FIG. 16 shows a collar assembly 180 having separate halves which are clamped around the syringes 132 using a quick latching attachment 182. FIG. 17 shows a collar assembly 190 formed as a clamshell design. The top section 191 is pivoted open, the syringes are installed, and then the top section is closed and snapped shut. A latch 192 holds the top section 191 and the bottom section 193 together around the syringes. The latch 192 may be releasable, or it may be of the type that cannot be opened or released after it is snapped closed. The collar assembly 190 is preferably made of a flexible material, or has a hinge section 195, to allow it to move from the open position to the closed position.

FIGS. 18-23 show another alternative syringe fixturing or holding system. As shown in FIG. 18, the alternative syringe assembly or system 200 uses two syringes 132, which may be standard syringes in current use. Each syringe 132 has a flange 134 and a plunger 135 which can slide into the body of the syringe to deliver an injectant. The syringes 135 are formed into the assembly using a collar 202 and a manifold 204.

Referring to FIG. 19, the collar 202 has an upper collar section 210 attached to a lower collar section 212 by a hinge 220. Each of the collar sections 210 and 212, in the embodiment shown, has a pair of connected syringe flange housings 214. Each syringe flange housing 214 has an end opening 216 and a radiused intersection 218. Referring now to FIGS. 18 and 19, the syringes 132 are placed into the collar 202 by initially hinging or pivoting apart the upper and lower collar sections 210 and 212, as shown in FIG. 19. The collars 202 of the syringes 132 are placed into the flange housings 214, with the ends or tips 135 of the flanges 134 extending through the openings 216, as shown in FIG. 19. The collar sections 210 and 212 are then brought together by hinging or pivoting movement about the hinge 220. A snap or closure 222 opposite to the hinge 220 secures the collar sections 210 and 212 together, thereby holding the syringes 132 in place via the flanges 134 of the syringes.

The collar 202 is preferably an integral molded plastic unit, with the hinge 220 simply formed via a thin section of material. However, multiple piece collar designs may also be used, with a separate hinge 220 attached to the collar sections 210 and 212. While the embodiment shown has openings 216 through which the flange tips 135 extend, the openings 216 may be omitted. Of course, the collar 202 may also be provided with additional flange housings 214, if three or more syringes are desired. The radiused intersections 218 form a clearance opening around the plungers 135, when the collar 202 is in the closed position, shown in FIG. 18, so as not to interfere with movement of the plungers.

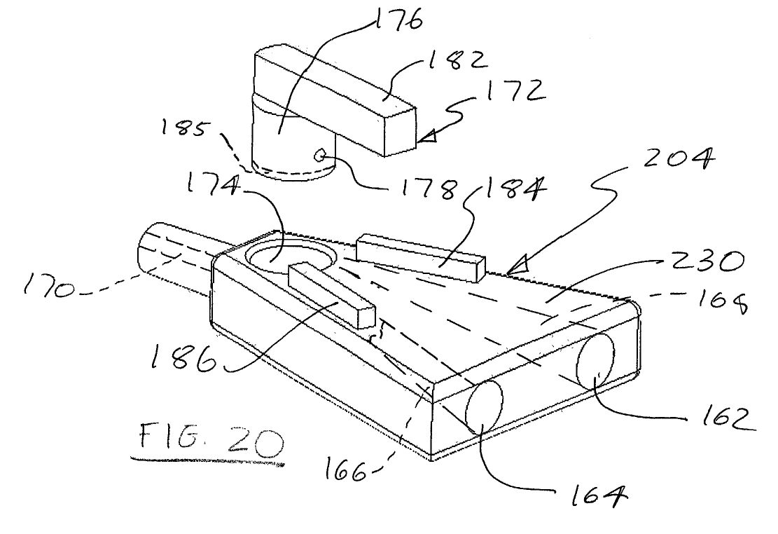

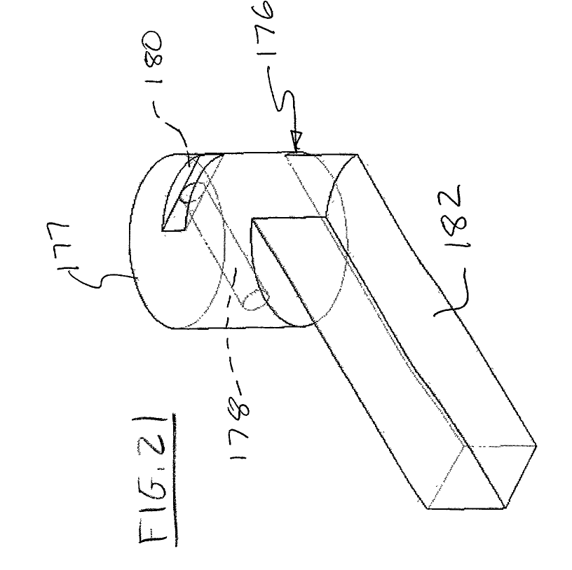

Turning now to FIG. 20, the manifold 204 includes a body 230, inlets 162 and 164, passageways 166 and 168, and an outlet 170, similar to the embodiment shown in FIG. 14 and described above. However, the manifold 204 shown in FIG. 20 also has a valve assembly 172 including a valve body 176 pivotably installed within a valve opening 174 in the body 230. In addition, first and second lever stops 184 and 186 are provided on the body 230.

As shown in FIG. 21, the valve body 176 has a body duct 178 extending centrally through a cylindrical barrel section 177. A slot 180 is provided at the front end of the barrel 177. The body duct 178 extends from the back end of the barrel section 177 and joins into a central location of the slot 180. A lever 182 is attached to or part of the barrel section 177.

The valve body 176 is installed within the body 230 via a snap ring 185 or similar securing feature, which prevents separation of the valve body 176 from the manifold body 230, yet allows the valve body to pivot between the first and second lever stops 184 and 186.

Referring to FIG. 20-23, with the valve body in the first position shown in FIG. 22, the body duct 178 in the valve body 176 is aligned with the first passageway 176 leading to the first syringe. The outlet 170 in the manifold body 230 leading into the needle 174 is not directly aligned with the body duct 178. However, the outlet 170 is aligned with the slot 180 on the valve body. Consequently, a fluid path is established from the first syringe into the passageway 166, through the body duct 178, into the slot 180, through the outlet 170 and then into the needle 174. At the same time, the second passageway 168 connecting to the second syringe is isolated by the valve body. Consequently, back-flow from the first syringe into the second syringe is prevented.

FIG. 23 shows the syringe assembly 200 with the valve body 176 in the second position, for delivering an injectant from the second syringe through the needle 174, while isolating the fluid pathway from the first syringe, to avoid any back-flow.

In use, syringes are placed into the collar 202, as shown in FIG. 19. The flanges 134 of the syringes extend into the flange housings 214 of the lower or first collar section 212. Preferably, the tips 135 of the flanges 134 extend out of the openings 216 in the collar sections 210 and 212. The upper or second collar section 210 is then moved from the open position, shown in FIG. 19, to the closed position, shown in FIG. 18. The collar sections 210 and 212 are held together by a snap 222. The snap 222 may be a permanent one-way snap, preventing separation of the collar sections 210 and 212 after the snap 222 is engaged. The permanent snap 222 is preferably for single-use applications, wherein the components of the system 200 are disposed of after a single use. Alternatively, the snap may be releaseable, allowing reuse of the components. The upper and lower collar sections 210 and 212 are sized and dimensioned to fit securely around the flanges 134 of the syringes 132. The collar 202 may be provided in different sizes and shapes for use in syringes having flanges 134 of different sizes and shapes. The syringes 132 may be prefilled with first and second liquid injectants. Alternatively, the syringes may be filled after they are joined together by the collar 202.

Turning to FIG. 18, the end tubes of the syringes 132 are placed into the inlets 162 and 164 of the manifold 204. The inlets 162 and 164 are advantageously dimensioned to provide a friction, push-on fit with the syringes. A needle 174 is placed onto the outlet 170 of the manifold 204, preferably using standard needle fittings. The lever 182 is placed in the first position, as shown in FIG. 22. The lever 182 is against the first lever stop 184. The assembly 200 is now positioned to deliver injectant from the first syringe 132A. The needle 174 is placed into the injection site. The plunger of the first syringe 132A is pressed in. Injectant from the first syringe 132A flows through the first inlet 162, through the first passageway 166, into the body duct 178 of the valve body 176, through the slot 180 and the outlet 170, through the needle 174 and into the injection site. With the valve body 176 in the position in FIG. 22, the liquid injectant flowing through the manifold 204 cannot mix with or flow into the second passageway 168 or the second syringe 132B, because the second passageway 168 is blocked by the cylindrical barrel section 177 of the valve body.

To deliver injectant form the second syringe 132B, the lever 182 is moved from the first position shown in FIG. 22, into the second position, shown in FIG. 23. In the second position, the lever 182 is against the second lever stop 186. With the lever 182 and valve body 176 in the second position, the plunger of the second syringe 132B is pressed in. Injectant flows from the second syringe 132B into the second inlet 164, through the second passageway 168, into the body duct 178 (which is aligned with the second passageway 168), into the slot 180, through the outlet 170, through the needle 174 and into the injection site. As there is flow path connecting the passageways 166 and 168 at any time, mixing of the liquid injectants via back-flow is prevented. As the collars do not come into contact with the injectant, they can be readily reused, regardless of the design.

The methods described above may also be used with electrosurgical techniques, such as described in U.S. Pat. Nos. 5,695,495 and 6,293,944, incorporated herein by reference, or with ultrasound image-guided techniques to locate the injection site, or both.

Thus, novel methods and devices have been shown and described. Various substitutions of steps and components may of course be made without departing from the spirit and scope of the invention. The invention, therefore, should not be limited, except by the following claims, and their equivalents.

1. A syringe system comprising:
a collar having first and second positions for receiving flanges of first and second syringes; and
a manifold having first and second inlets connectable to first and second syringes held in the dollar assembly, respectively, and the manifold including a single outlet having a needle fitting; and
a valve assembly on the manifold moveable to connect one of the first and second inlets to the outlet.

2. The syringe system of claim 1 with the valve assembly including a valve body having a body duct extending through a cylindrical barrel section and joining into a slot on the barrel section.

3. The syringe system of claim 1 with the first and second collar sections each having at least two flange housings adapted to fit around a flange on the syringes.

4. A syringe system comprising:
a collar assembly having first and second positions adapted to receive first and second syringes; and
a manifold having first and second inlets connectable to first and second syringes held in the collar assembly, respectively, and the manifold further including a single outlet joining into the first and second inlet, and a needle fitting around the outlet.

5. The system of claim 4 with the collar assembly including first and second flange recesses adapted to receive first and second flanges on the first and second syringes.

6. The system of claim 5 further comprising first and second arms pivotably attached to the plate.

7. The system of claim 6 further comprising a detent on the plate, for holding the first and second arms into a closed position.

8. The system of claim 6 further comprising a spring on the plate biasing the first and second arms into a closed position.
